(19) United States
(12) Patent Application Publication
(10) Pub. No.: US 2003/0077232 A1
(43) Pub. Date: Apr. 24, 2003

(54) Composition

(75) Inventors: Victoria Cromwell, Merseyside (GB); Peter Freunscht, Merseyside (GB); Peter John Hall, Merseyside (GB); David Thomas Littlewood, Merseyside (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/225,857

(22) Filed: Aug. 22, 2002

(30) Foreign Application Priority Data
Aug. 24, 2001 (EP) 01307269.9
Dec. 11, 2001 (EP) 01310338.7

(51) Int. Cl. A61K 9/68; A61K 7/16

(52) U.S. Cl. 424/48; 424/49

(57) ABSTRACT

Oral composition comprising an alkyl hydroxybenzoate represented by formula I

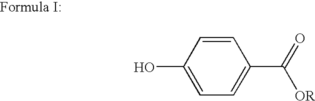

wherein R represents a straight chain alkyl group comprising at least eight carbon atoms.

The present invention relates to an oral composition comprising an alkyl hydroxybenzoate.

Alkyl hydroxybenzoates (parabens) are known in the art where the alkyl group is methyl. For example, methyl hydroxybenzoate is mentioned, albeit fleetingly, for use in medicinal and oral care preparations as a preservative (WO 00/09507 and WO 00/69401).

In addition, U.S. Pat. No. 5,094,841 (Fine) discloses the use of heptyl paraben as a preservative in an oral care formulation. However, it also states that the preferred preservatives are methyl and propyl paraben and only ever states that they may be included in small amounts (0.1%) to provide a preservative effect.

EP-A2-0 161 898 (Unilever) discloses that an oral composition can comprise non-cationic antimicrobial agents selected from the esters of p-hydroxybenzoic acid, especially the methyl, ethyl, propyl, isopropyl, butyl, isobutyl, hexyl, heptyl and benzyl esters.

The longer chain materials are less likely to be used in a composition such as an oral care composition because long alkyl chain lengths are typically difficult to formulate due to their hydrophobicity. This is why the majority of the prior art teaches the C1 to C4 parabens and not the C6 or C7 parabens.

We have found that there exists compounds which exhibit surprisingly high antibacterial efficacy and are not disclosed for use in oral compositions in the prior art.

### STATEMENT OF INVENTION

Accordingly, the invention provides oral composition comprising an alkyl hydroxybenzoate represented by formula 1

Formula 1:

wherein R represents a straight chain alkyl group comprising

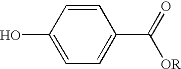

at least eight carbon atoms.

### DESCRIPTION OF INVENTION

The alkyl group of the compound according to formula 1 is a straight chain alkyl comprising at least eight carbon atoms. Preferably, the alkyl group comprises no more than 30 carbon atoms. More preferably the alkyl group comprises from 8 to 15 carbon atoms, especially from 8 to 10 and most preferably 8.

Further, the alkyl group may be substituted or unsubstituted.

Preferred alkyl groups include octyl, nonyl, decyl, undecyl and dodecyl. More preferably the alkyl group is n-octyl. Such compounds may be made by simple esterification of 4-hydroxybenzoic acid with the respective alcohol. Such a process is a simple step for the man skilled in the art to carry out.

The most preferred antimicrobial agent is n-octyl parahydroxy benzoic acid because it has the greatest antimicrobial effect against the commonly present oral microflora. Many of the other parabens are effective only against certain of these bacteria or are less effective against the same range of microflora.

The compound according to formula 1 is preferably present in an amount such that an antibacterial effect can be provided. In practice this ranges from 0.15 to 30% by weight of the composition according to the invention. Preferably, in an amount ranging from 0.2 to 20% by weight and even more suitably from 0.25 to 10% by weight. Most preferably the amount of compound according to formula 1 ranges from 1.0 to 2.5% by weight of the composition.

The composition according to the invention may also comprise a divalent metal salt. Preferably, the divalent metal salt is a salt selected from the group consisting of zinc- and stannous salts such as zinc citrate, zinc sulphate, zinc glycinate, sodium zinc citrate, stannous pyrophosphate and mixtures thereof. The preferable divalent metal salt is zinc citrate.

Suitably, the amount of divalent metal salt ranges from 0.01 to 10% by weight of the composition, preferably from 0.05 to 5% by weight, more preferably from 0.1 to 2% by weight and especially preferably from 0.3 to 0.9% by weight of the composition.

The oral composition according to the invention comprise further ingredients which are common in the art, such as:

antimicrobial agents, e.g. Triclosan, chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition.

humectants such as glycerol, sorbitol, propyleneglycol, xylitol, lactitol etc.;

binders and thickeners such as sodium carboxymethyl-cellulose, xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

Embodiments according to the invention shall now be discussed with reference to the following non-limiting examples.

### EXAMPLE 1

The following method is used to assess the antimicrobial efficacy of the agents according to formula 1.

The seed stock of the bacterial strains, E. cloacae, A. naeslundii, S. sanguis (facultative anaerobes) and F. nucleatum and V. parvula (obligate anaerobes) is stored frozen in 1.5 ml aliquots. From the stock, an appropriate dilution of bacteria is added to BHI (dilution 1:500 for E. cloacae; dilution 1:200 for A. naeslundii; dilution 1:100 for S. sanguis; dilution 1:20 for F. nucleatum; and dilution 1:20 for V. parvula). For the two obligate anaearobic strains, F. nucleatum and V. parvula, the BHI medium is supplemented with Oxyrase (100 μl/5 ml). Oxyrase for Broth is a sterile enzyme additive which is used to produce anaerobic conditions in a wide variety of bacteriological broth medium. The cells in the BHI broth are added to 96 well plates at a volume of 180 μl/well. The compounds to be tested are added to the wells (20 μl/well) to give final assay concentrations over the desired range. The plates are incubated at 37° C. for specific period of time, determined separately for each bacterial culture. After the incubation period the optical density is measured using a Bio-Tek EL 340 Microplate Biokinetics® reader. For studies carried out with Alamar Blue® to monitor the growth of the cultures, fluorescence is measured using a Tecan Spectrafluor® fluorescence plate reader.

In order to establish a correlation between absorbance at 550 nm and cell density, a sequence of dilutions for each of the five organisms was made from a culture derived from the original stock vial. The facultative anaerobic cultures were incubated at 37° C. at a shaker setting of 250 rpm. The anaerobic cultures were incubated in Oxyrase® broth at 37° C. without shaking. After the incubation period, a serial dilution series covering the range of 10 to 1200 was made. Dilution samples were read on the Bio-Tek Biokinetics® plate reader at 550 nm.

Data will be expressed as Percent of Control. Positive controls (no sample) will be run with culture in the presence of 1.0% DMSO. Negative controls (no culture) will be run with media in the presence of 1.0% DMSO. Additionally, standard compounds (Chlorhexidine acetate, and Cetyl pyridinium chloride) may also be employed as Reference controls.

Percent of control is calculated by the following formula:

$$\text{\% of Control} = \frac{[ Sample - Negative Control ]}{[ Positive - Negative Control ]} \times 100$$

Calculation of MIC values were carried out using the XL fit program after plotting the dose response curves.

### EXAMPLE 2

The antimicrobial efficacy (MIC values) of agents according to formula 1 and also some agents which do not form part of the invention are as follows:

| | Actinornyces | Fusobacterium | Streptococcus | Veilonella |
|---|---|---|---|---|
| | naeslundii | nucleatum | sanguis | parvula |
| R of | | | | |
| Formula 1 | | | | |
| Comparative | | | | |
| examples | | | | |
| methyl | >128 | >128 | 128 | 128 |
| paraben | | | | |
| ethyl | >128 | >128 | >128 | >128 |
| paraben | | | | |
| propyl | >128 | 128 | 128 | >128 |
| paraben | | | | |
| isopropyl | >128 | 94 | 128 | >128 |
| paraben | | | | |
| butyl | >128 | 32.5 | 128 | 128 |
| paraben | | | | |
| isobutyl | >128 | 42.7 | 128 | 128 |
| paraben | | | | |
| benzyl | 128 | 42 | 128 | 42 |
| paraben | | | | |
| heptyl | 42 | 42 | 14.2 | 42 |
| paraben | | | | |
| Example | | | | |
| according to | | | | |
| the | | | | |
| invention | | | | |
| n-octyl | 14 | 14.2 | 2.7 | 42 |

The agent according to Formula 1 where R is n-octyl can be seen to have a greater antimicrobial efficacy and greater spectrum of activity against oral bacteria than any of the other parabens.

### EXAMPLE 3

The following is a formulation according to the present invention. It is made by known processes.

| Ingredient | % w/w |
|---|---|
| 70% aq. sorbitol | 45.0 |
| Saccharin | 0.2 |
| Polyethylene glycol | 2.0 |
| Titanium dioxide | 1.0 |
| Sodium fluoride | 0.32 |
| Thickening silica | 9.0 |
| Abrasive silica | 10.0 |
| SLS | 1.6 |
| Sodium carboxymethylcellulose | 0.8 |
| Flavour | 1.0 |
| Zinc citrate trihydrate | 0.75 |
| n-Octyl paraben | 1.0 |
| Water | to 100 |

1. Oral composition comprising an alkyl hydroxybenzoate represented by formula 1 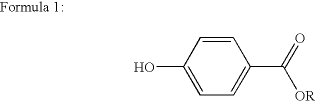
wherein r represents a straight chain alkyl group comprising at least eight carbon atoms.

2. Composition according to claim 1, wherein R represents a straight chain alkyl group comprising from eight to ten carbon atoms.

3. Composition according to claim 1, wherein R is n-octyl.

4. Composition according to claim 1 and comprising an orally acceptable carrier.

5. Composition according to claim 1 and selected from the group consisting of pastes, gels, foams, liquids, powders, chewing gums, wherein the composition is suitable for use in dental care.

6. Composition according to claim 1, wherein the composition comprises from 0.2 to 5% by weight of the alkyl hydroxybenzoate.

7. Composition according to claim 1 comprising an antimicrobially effective amount of a divalent metal ion source.

8. Composition according to claim 7, wherein the divalent metal is zinc.
